(19) United States
(12) Patent Application Publication
(10) Pub. No.: US 2003/0225302 A1
(43) Pub. Date: Dec. 4, 2003

(54) Aromatic amides, their preparation process and their use as pesticides

(76) Inventors: Jacques Demassey, Montevrain (FR); Robert Peek, Bondy (FR); Antonio Ugolini, Massy (FR); John Weston, Maisons Lafitte (FR)

Correspondence Address: FROMMER LAWRENCE & HAUG, 745 FIFTH AVENUE- 10TH FL., NEW YORK, NY, 10151, US

(21) Appl. No.: 10/429,112

(22) Filed: May 2, 2003

Related U.S. Application Data
(63) Continuation of application No. 09/446,879, filed on Dec. 28, 1999, now abandoned.

(30) Foreign Application Priority Data
Jul. 2, 1997 (FR) 97/08334

(51) Int. Cl. A01N 37/18; C07C 233/64

(52) U.S. Cl. 564/171; 514/617

(57) ABSTRACT

Aromatic amides, their preparation process and their use as pesticides

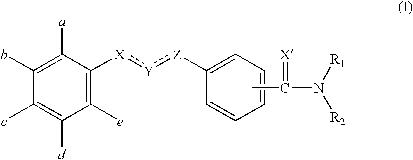

Compounds of formula (I):

in which

a, b, c, d and e identical to or different from one another, represent a hydrogen atom, a halogen atom, an alkyl, alkenyl or alkynyl, O-alkyl, O-alkenyl or O-alkynyl, S-alkyl, S-alkenyl or S-alkynyl, each radical containing up to 8 carbon atoms, optionally substituted by one or more halogen atoms, a SF₅, C N, NO₂ or NH₂ radical, the substituents a, b, c and d being able to form between themselves rings which either carry or do not carry one or more heteroatoms and being able to be substituted;

X and Y or Y and Z identical or different, represent a radical: —HC═CH—, —(CH₃)C═CH—, -(Hal)C═C—, ═C(f)(g), or ═C═Cx¹x²,

Hal represents a halogen atom:

X¹ represents a hydrogen atom or a halogen atom, in which f and g, identical to or different from each other, represent a hydrogen atom, a halogen atom, a free, etherified or esterified hydroxyl radical, an alkyl radical, containing up to 8 carbon atoms, optionally substituted by one or more halogen atoms, or represent a C═O radical, an oxygen atom;

X′ represents an oxygen or sulphur atom,

R¹ and R² identical to or different from each other, represent a hydrogen atom, a linear, branched or cyclic, saturated or unsaturated alkyl, CO-alkyl or CO₂-alkyl radical, containing up to 8 carbon atoms, optionally interrupted by one or more heteroatoms, optionally substituted, or an optionally substituted aryl or heteroaryl radical, the —C(X′)—NR¹R² chain being in meta or para position, and

dotted lines representing one or more optional double bonds,

in all their possible isomeric forms as well as their mixture.

The compounds of formula (I) have pesticidal properties.

The present invention relates to aromatic amides, their preparation process and their use as pesticides.

A subject of the present invention is the compounds of formula (1):

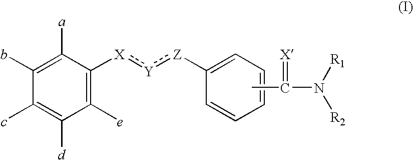

in which

a, b, c, d and e identical to or different from one another, represent a hydrogen atom, a halogen atom, an alkyl, alkenyl or alkynyl, O-alkyl, O-alkenyl or O-alkynyl, S-alkyl, S-alkenyl or S-alkynyl, each radical containing up to 8 carbon atoms, optionally substituted by one or more halogen atoms, a SF₅, C N, NO₂ or NH₂ radical, the substituents a, b, c and d being able to form between themselves rings which either carry or do not carry one or more heteroatoms and being able to be substituted;

X and Y or Y and Z identical or different, represent a radical: —HC═CH—, —(CH₃)C═CH—, -(Hal)C═C—, C(f)(g), or ═C═C X¹X²,

Hal represents a halogen atom;

X¹ represents a hydrogen atom or a halogen atom;

f, g, identical to or different from each other, represent a hydrogen atom, a halogen atom, a free, etherified or esterified hydroxyl radical, an alkyl radical, containing up to 8 carbon atoms, optionally substituted by one or more halogen atoms, or represent a C═O radical, an oxygen atom;

X′ represents an oxygen or sulphur atom;

R¹ and R² identical to or different from each other, represent a hydrogen atom, a linear, branched or cyclic, saturated or unsaturated alkyl, CO-alkyl or CO₂-alkyl radical, containing up to 8 carbon atoms, optionally interrupted by one or more heteroatoms, optionally substituted, or an optionally substituted aryl or heteroaryl radical, the —C(X′)—NR¹R² chain being in meta or para position, and

dotted lines representing one or more optional double bonds;

in all their possible isomeric forms as well as their mixture.

By compound of formula (I) are designated all the possible geometric isomers and stereo-isomers taken individually or in a mixture.

In the definition of substituents:

alkyl preferably represents a methyl, ethyl, propyl, isopropyl, butyl, isobutyl, n-pentyl, isopentyl, cyclo-propyl, cyclobutyl or cyclopentyl radical, alkenyl prefer-ably represents a vinyl, 1-propenyl, 2-methyl, 2-propenyl or isoprenyl radical,

alkynyl preferably represents an ethynyl, 1-propynyl, 2-propynyl or pent-2-ene-4-ynyl radical,

halogen preferably represents a fluorine, chlorine, bromine or iodine atom, preferably a fluorine, chlorine or bromine atom,

aryl preferably represents a carbocyclic aromatic group containing 4 to 10 carbon atoms, in particular a phenyl or naphthyl radical,

a heterocyclic radical is preferably a heteroaryl radical or a saturated or unsaturated 3 to 8 membered ring comprising one, two, three or four heteroatoms from the group consisting of N, O and S. heterocyclic is preferably a 3 to 7 membered aromatic ring comprising one, two, three or four heteroatoms from the group consisting of N, O and S, particularly preferred thienyl, furyl, pyrrolyl, pyrazolyl, pyridyl, pyrazinyl, pyrimidinyl, pyridazinyl, isothiazolyl, isoxazolyl, oxazolyl, thiazolyl, oxaidazolyl and tetrazinyl

Particularly preferred heterocyclic radicals are thienyl, furyl, pyrannyl, pyrrolyl, 2H-pyrrolyl, imidazolyl, pyrazolyl, pyridyl, pyrazinly, pyrimidinyl, pyridazinyl, isothiazolyl, isoxazolyl, furazannyl, thiazolyl, oxazolyl, pyrrolidinyl, pyrrolinyl, imidazolidinyl, pyrazolinyl, piperidyl, piperazinyl, morpholinyl, thiazinyl, tetrazinyl, oxathiolanyl or thiadiazinyl radicals.

When a radical is substituted, it is preferably substituted by one or more substituents chosen in particular from halogen atoms, alkoxy radicals containing up to 8 carbon atoms, or methylenedioxy, difluoromethylenedioxy, tetrafluoro ethylenedioxy, cyano, nitro, cyanato, thiocyanato, pentafluorothio or fluorosulfonyl groups.

Etherified or esterified preferably means etherified with a linear or branched C₁-C₈-alkyl group or esterified with a (C₁-C₈)-carboxylic acid.

If any of the substituents a-e form a ring it is preferably a 4 to 8 membered ring which is preferably monounsaturated (due to fusion with the phenyl group) and is carbocyclic or contains preferably one or two heteroatoms from the group consisting of N, O and S.

More particularly, a subject of the invention is the compounds of formula (I) in which the C(X′)—NR¹R² chain is in para position, those in which X′ represents an oxygen atom, those in which R¹ represents a hydrogen atom, those in which R² represents an optionally substituted aryl radical and in particular a 2-methylphenyl radical, those in which R² represents an alkyl radical containing up to 8 carbon atoms, linear or branched, those in which at least one of substituents a, b, c and d represent a halogen atom, and in particular those in which the halogen is chlorine or bromine.

More particularly, a subject of the invention is the compounds of formula (I) in which b, c and d each represent a chlorine or hydrogen atom, those in which a and e each represent a hydrogen atom, those in which Z represents a —CH₂— radical, those in which X and Y together represent a radical, those in which Y and Z together represent a radical:

—FC═CH—, —(CH₃)C═CH— or —HC═CF—.

More particularly, a subject of the invention is the compounds the preparation of which is given hereafter in the experimental part.

A subject of the invention is also a process wherein a compound of formula (II):

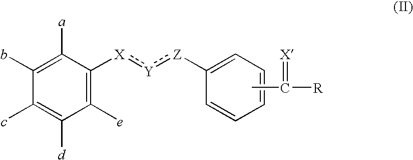

in which a, b, c, d, e, X, Y, Z and X′ and the dotted lines retain the same meaning as previously and R represents a hydroxy radical, a halogen atom, an alkoxy radical containing up to 4 carbon atoms, or a —P(O)(φ)NHφ group in which Y represents a phenyl group, is subjected to the action of a compound of formula (III):

HNR¹R² (III)

in which R₁ and R₂ retain their previous meaning in order to obtain the corresponding compound of formula (I).

Among the preferred products there can be cited quite particularly:

4-[3-(3,4-dichlorophenyl)-2-fluoropropyl]-N-(2-methyl-phenyl)-benzamide

4-[3-(3,4-dichlorophenyl)-2-fluoropropyl]-N-(1,2-dimethyl-propyl)-benzamide

4-[3-(3,4-dichlorophenyl)-2-fluoropropyl]-N-(4-fluoro-2-methylphenyl)-benzamide

4-[3-(3,4-dichlorophenyl)-3-fluoropropyl]-N-(2-methyl-phenyl)-benzamide

4-[3-(3,4-dichlorophenyl)-2-propenyl]-N-(2-methylphenyl)-benzamide

4-[3-(4-bromo-2-methylphenyl)-2-propenyl]-N-(2-methyl-phenyl)-benzamide

4-[3-(3,4-dichlorophenyl)-2-fluorobutyl]-N-(2-methyl-phenyl)-benzamide

4-[3-(3,4-dichlorophenyl)-2-butenyl]-N-(2-methylphenyl)-benzamide

4-[3-chloro-3-(3,4-dichlorophenyl]-2-propenyl]-N-(2-methylphenyl)-benzamide

4-[3-(3,4-dichlorophenyl)-2-butenyl]-N-(1,2-dimethyl-propyl)-benzamide

4-[3-(3,4-dichlorophenyl)-2-fluoro-2-propenyl]-N-(2-methylphenyl)-benzamide

4-[3-(3,4-dichlorophenyl)-2-fluoro-2-propenyl]-N-ethyl-N-(2-methylphenyl)-benzamide

4-[3-(3,4-dichlorophenyl)-2-pentenyl]-N-(2-methylphenyl)-benzamide.

The products of formula (I) thus obtained can be, if appropriate, separated into their optically active isomers.

Separation of the isomers can be carried out according to methods known to a person skilled in the art for example by crystallization or by chromatography.

The amidification reaction is in general carried out at a temperature comprised between −25 C and 150 C in an anhydrous and aprotic solvent such as ether, dichloro-methane, toluene or benzene.

The products of formula (II) used as starting products are new and are in themselves a subject of the present invention, they can be prepared according to the processes described below in the experimental section.

The compounds of formula (I) can be used to combat harmful organisms such as arthropods, for example insects and acaridae, and helminths, for example nematodes, or molluscs, for example slugs. Therefore a subject of the present invention is a process for combating arthropods and/or helminths and/or molluscs, which comprises the administration to the arthropods and/or helminths and/or molluscs, or to their environment, of a quantity of a compound of formula (I) which is sufficient to destroy the harmful organism. Also a subject of the present invention is a process for combating and/or eradicating infestations by arthropods and/or helminths and/or molluscs in animals (including humans) and/or plants (including trees) and/or stored products, which comprises the administration to the animal or to the locality of an effective quantity of a compound of formula (I). A subject of the invention is also the compounds of formula (I) to be used in human and veterinary medicine, in public health and/or in agriculture for combating harmful arthropods and/or helminths.

The compounds of formula (I) are particularly valuable in the protection of standing crops, forage crops, crops in plantations, in greenhouses, in orchards and in vineyards, of ornamental plants and trees in plantations and forests, for example cereals (such as corn, wheat, rice and sorghum), cotton, tobacco, vegetables and salad vegetables (such as beans, cabbages, cucurbitaceae, lettuces, onions, tomatoes and peppers), food crops (such as potatoes, sugar beet, peanuts, soya and oilseed rape), sugar cane, meadows and forage crops (such as corn, sorghum and alfalfa), plant-ations (such as those producing tea, coffee, cocoa, banana, palm oil, coconut, rubber and spices), orchards and tree plantations (such as those producing stone fruits and pome fruit, citrus fruits, kiwis, avocados, mangoes, olives and walnuts), vines, ornamental plants, flowers and bushes in greenhouses and in gardens and parks, forest trees (both deciduous and evergreen) in forests, plantations and nurseries.

They are also valuable in the protection of timber (standing, felled, processed, stored or in buildings) against attack from wood wasps (for example Urocerus) or coleopterous insects (for example scolytidae, platypodidae, lyctidae, bostrichidae, cerambucidae and anobiidae) and termites.

They are used in the protection of stored products, such as grains, fruits, nuts, spices and tobacco, whether whole, ground or converted into products, against attack from mites, coleopterous insects and weevils. They also protect stored animal products such as skins, furs, wool and feathers, in natural or processed form (for example rugs or textile materials) against attack from mites and coleopt-erous insects, similarly meat and fish against attack from coleopterous insects and flies.

The compounds of general formula (I) are particularly useful for combating arthropods, helminths or molluscs, which are harmful to man and domestic animals, or spread or are carriers of diseases affecting the latter, for example those described above, more particularly in the field of combating ticks, mites, lice, fleas, midges and flies which cause bites and are harmful.

The invention also relates in particular to the use of the compounds of formula (I) as defined previously, as pesticides in particular as insecticides, aracides and nematicides in the protection of crops in particular rice and cotton crops, or for the treatment of premises for storing products of the said crops and in particular as insecticides and aracides in domestic or public premises.

The compounds of formula (I) can be used to these ends by the application of the compounds as they are, or in a diluted form in a known manner in the form of dips, sprays, mists, lacquers, foams, powders, dusting powders, aqueous suspensions, pastes, gels, shampoos, ointments, combustible solids, spray pads, combustible coils, baits, food additives, wettable powders, granules, aerosols, emulsifiable concentrates, oily suspensions, oily solutions, pressurized sprays, impregnated articles, lotions or other standard compositions well known to a person skilled in the art. Concentrates for dips are not used as they are, but diluted with water, and the animals are immersed in a tank containing the dip. Sprays can be applied by hand, or with the help of a spray lance or frame. The animal, the ground, the plant or the surface can be saturated with the spray using a high volume application, or coated superficially with the spray by application in a small or ultra-small volume. Aqueous suspensions can be applied to the animal in the same manner as sprays and dips. Dusting powders can be distributed via a powder applicator or, in the case of animals, be incorporated in perforated bags fixed to trees or poles. Pastes, shampoos and ointments can be applied by hand or spread on the surface of an inert material against which the animals rub themselves and thus transfer the product onto their skin. Lotions are distributed as a low-volume dose of liquid on the backs of animals, so that all or most of the liquid remains on the animals.

The compounds of formula (I) can be presented as compositions ready to use on plants, animals and surfaces or in the form of compositions which must be diluted before use, but both types of compositions contain a compound of formula (I) intimately mixed with one or more excipients or diluents. The excipients can be liquid, solid or gaseous, or can comprise mixtures of such substances, and the compound of formula (I) can be present in a concentration of 99 to 0.025% w/v, according to whether the composition does or does not need to be more diluted.

Dusting powders, powders and granules contain the compound of formula (I) intimately mixed with a pulverulent solid inert excipient, for example suitable clays, kaolin, bentonite, attapulgite, adsorbent carbon black, talc, mica, chalk, gypsum, tricalcium phosphate, powdered cork, magnes-ium silicate, vegetable excipients, starch and diatomaceous earths. These solid compositions are in general prepared by impregnating the solid diluents with solutions of the compound of formula (I) in volatile solvents, by evaporating the solvents and, if desired, grinding the products to obtain powders and, if desired, by granulating, compacting or encapsulating the products.

The sprays of a compound of formula (I) can comprise a solution in an organic solvent (for example those mentioned below) or an emulsion in water (dipping or spraying), pre-pared on site from an emulsifiable concentrate (also called oil miscible with water), which can also be used for dipping. The concentrate preferably comprises a mixture of the active ingredient, with or without organic solvent, and one or more emulsifiers. Solvents can be present within broad limits, but preferably in a quantity of 0 to 90% w/v of the composition, and can be chosen from kerosene, ket-ones, alcohols, xylene, aromatic naphtha and other solvents known for use in composition. The concentration of the emulsifiers can vary within broad limits, but is preferably in the range of 5 to 25% w/v, and the emulsifiers are advantageously non-ionic surfactants, in particular polyoxy-alkylenic esters of alkylphenols and polyoxyethylenic deriv-atives of hexitol anhydrides, or anionic surfactants, in particular sodium laurylsulfate, fatty alcohol ether sulfates, the sodium and calcium salts of alkylaryl sulfonates and alkylsulfo-succinates.

Cationic emulsifiers are in particular benzalkonium chloride and quaternary ammonium ethylsulfates.

Amphoteric emulsifiers are in particular carboxy-methylated oleic imidazoline and alkyldimethyl-betaines.

Vaporization wicks normally comprise a mixture of cotton and cellulose compressed into a pad, for example of approximately 32 mm by 22 mm by 3 mm, treated by means of a quantity reaching preferably 0.3 ml of a concentrate which contains the active ingredient in an organic solvent and optionally an anti-oxidant, a coloring agent and a perfume.

The insecticide is vaporized preferably by a heat source, such as an electrically-powered heating device for wicks.

The combustible solids normally comprise sawdust and a binder mixed with the active ingredient and used fashioned into strips (usually in coils). A coloring agent and a fungicide can also be added.

The wettable powders contain an inert solid excipient, one or more surfactants, and optionally stabilizers and/or anti-oxidants.

The emulsifiable concentrates comprise emulsifying agents and often an organic solvent, such as kerosene, ketones, alcohols, xylenes, aromatic naphtha and other known solvents.

The wettable powders and emulsifiable concentrates normally contain 5 to 95% by weight of the active ingredient and are diluted, for example with water, before use.

The lacquers comprise a solution of the active ingredient in an organic solvent, together with a resin and optionally a plasticizer.

The dips can be prepared not only from emulsifiable concentrates, but also from wettable powders, dips based on soap and aqueous suspensions comprising a compound of formula (I) intimately mixed with a dispersing agent and one or more surfactants.

The aqueous suspensions of a compound of formula (I) can comprise a suspension in water together with a suspension, stabilization or other agent. The suspensions or solutions can be applied as they are or in a form diluted in a known manner.

The ointments (or greases) can be prepared from vegetable oils, synthetic esters of fatty acids or lanolin, together with an inert base such as soft paraffin. A compound of formula (I) is preferably distributed uniformly throughout the mixture, in solution or in suspension. The ointments can also be obtained from emulsifiable concent-rates by dilution of the latter in an ointment base.

The pastes and shampoos are also semi-solid compos-itions in which a compound of formula (I) can be present as a uniform dispersion in a suitable base, such as soft or liquid paraffin, or in a non-fat base with glycerol, a glue or a suitable soap. Since the ointments, shampoos and pastes are normally applied without any other dilution, they must contain the appropriate percentage of the compound of formula (I) required by the treatment.

The aerosol sprays can be prepared in the form of a simple solution of the active ingredient in the aerosol pro-pellant and a co-solvent, such as a halogenated alkane and the above-mentioned solvents, respectively. The lotion compositions can be presented as a solution or suspension of a compound of formula (I) in a liquid medium. A bird or mammalian host can also be protected against infestation by acarid ectoparasites by wearing a manufactured product packed in suitably molded plastic which is impregnated with a compound of formula (I). These manufactured products include collars, ear tags, bands, sheets and ribbons suitably fixed to the appropriate part of the body. Advant-ageously, the plastic material is a poly(vinyl chloride).

Therefore, a subject of the invention is in particular a composition comprising:

a) a compound of formula (I) as defined previously,

b) inert, preferably customary, excipients suitable for use as pesticides of the said product of formula (I),

a composition comprising:

a) a compound of formula (I) as defined previously,

b) inert, preferably customary, excipients suitable for use in the veterinary field of the said product of formula (I),

and a compound of formula (I) as defined previously, for the implementation of a treatment method for the human or animal body characterized in that a pharmaceutically acceptable formulation of said compound is applied to said body.

The compounds of formula (I) are to be used in the protection and the treatment of plant species, in which case an effective insecticidal, acaricidal, molluscidal or nemat-ocidal quantity of the active ingredient is applied. The application dose varies with the chosen compound, the nature of the composition, the method of application, the type of plant, the density of plantation, the probable infestation, and various other factors, but in general a suitable applic-ation dose for agriculture is in the range of 0.001 to 3 kg per hectare, and preferably between 0.01 and 1 kg per hectare. Typical compositions for agricultural use contain between 0.0001% and 50% of a compound of formula (I) and advantageously between 0.1 and 15% by weight of a compound of formula (I).

The concentration of the compound of formula (I) for an application on an animal, in premises or in outside areas varies according to the chosen compound, the interval between treatments, the nature of the composition and the probable infestation, but, in general the compound must be contained in the applied composition in a quantity of 0.001 to 20.0% w/v, preferably 0.01 to 10% w/v. The quantity of compound deposited on an animal varies with the application method, the size of the animal, the concentration of the compound in the applied composition, the dilution factor of the composition and the nature of the composition, but is generally in the range of 0.0001% to 0.5% w/w, except for undiluted compositions, such as lotion compositions which are in general deposited at a concentration in the range of 0.1 to 20.0%, and preferably 0.1 to 10%. The quantity of compound to be applied to stored products is in general in the range of 0.1 to 20 ppm. Sprayings in areas can be carried out so as to obtain an initial average concentration of 0.001 to 1 mg of compound of formula (I) per m³ of treated area.

The ointments, greases, pastes and aerosols are usually applied at random, as described above and concentrations of 0.001 to 20% w/v of a compound of formula (I) in the applied composition can be used.

The compounds of formula (I) are particularly active against lipidoptera such as Spodoptera littoralis, Heliothis virescens, Plutella xylostella, against coleoptera such as Leptinotarsa decemlineata and Phaedon cochleariae.

The compounds of formula (I) are thus useful for combating arthropods, for example insects and acaridae, in any environment in which they are harmful, for example in agriculture, in breeding, in public health and in domestic environments.

Harmful insects are in particular memebers of the orders of coleoptera (for example Anobium, Ceuthorrhynchus, Rhynchophorus, Cosmopolites, Lissorhoptrus, Meligethes, Hypothenemus, Hylesinus, Acalymma, Lema, Psylliodes, Lept-inotarsa, Gonocephalum, Agriotes, Dermolepida, Heteronychus, Phaedon, Tribolium, Sitophilus, Diabrotica, Anthonomus or Anthrenus spp.), lepidoptera (for example Ephestia, Mamestra, Earias, Pectinophora, Ostrinia, Trichoplusia, Pieris, Laphygma, Agrotis, Amathes, Wiseana, Tryporyza, Diatraea, Sparganothis, Cydia, Archips, Plutella, Chilo, Heliothis, Spodoptera littoralis, Helrotuis virescens, Spod-optera or Tineola spp.), diptera (for example Musca, Aedes, Anopheles, Culex, Glossina, Simulium, Stomoxys, Haematobia, Tabanus, Hydrotaea, Lucilia, Chrysomyia, Callitroga, Dermat-obia, Gasterophilus, Hypoderma, Hylemyia, Atherigona, Chlorops, Phytomyza, Ceratitis, Liriomyza and Melophagus spp.), phthiraptera (Mallophaga, for example Damalina spp., and Anoplura, for example Linognathus and Haematopinus spp.), hemiptera (for example Aphis, Bemisia, Phorodon, Aeneolamia, Empoasca, Parkinsiella, Pyrilla, Aonidiella, Coccus, Pseudococcus, Helopeltis, Lygus, Dysdercus, Oxy-carenus, Nezara, Aleyrodes, Triatoma, Psylla, Myzus, Megoura, Phylloxera, Adelges, Nilaparvata, Nephrotettix or Cimex spp.), orthoptera (for example Locusta, Gryllus, Schistocerca or Acheta spp.), dictyoptera (for example Blattella, Periplaneta or Blatta spp.), hymenoptera (for example Athalia, Cephus, Atta, Solenopsis or Monomorium spp.), isoptera (for example Odontotermes and Reticulitermes spp.), siphonaptera (for example Ctenocephalides or Pulex spp.), thysanura (for example Lepisma spp.), dermaptera (for example Forficula spp.) and psocoptera (for example Perip-socus spp.) and thysanoptera (for example Thrips tabaci).

Harmful acaridae are in particular ticks, for example the members of the genera Bcophilus, Omithodorus, Rhipi-cephalus, Amblyomma, Hyalomma, Ixodes, Haemaphysalis, Derm-acentor and Anocentor, and acardiae and mites such as Acarus, Tetranychus, Psoroptes, Notoednes, Sarcoptes, Psor-ergates, Chorioptes, Eutrombicula, Demodex, Panonychus, Bryobia, Eriophyes, Blaniulus, Polyphagotarsonemus, Scuti-gerella and Oniscus spp.

Nematodes which attack plants and trees which are important in agriculture, forestry and horticulture, either directly or by spreading bacterial, viral, mycoplasmal or fungal diseases of plants, are in particular root node nematodes, such as Meloidogyne spp. (for example M. incog-nita); cyst nematodes, such as Globodera spp. (for example G. rostochiensis); Heterodera spp. (for example H. avenae); Radopholus spp. (for example R. similis); grassland nema-todes, such as Pratylenchus spp. (for example P. pratensis); Belonolaimus spp.: (for example B. gracilis); Tylenchulus spp. (for example T. semipenetrans); Rotylenchulus spp. (for example R. reniformis); Rotylenchus spp. (for example R. robustus); Helicotylenchus spp. (for example H. multi-cinctus); Hemicycliophora spp. (for example H. gracilis); Criconemoides spp. (for example C. similis); Trichodorus spp. (for example T. primitivus); tusk nematodes, such as Xiphinema spp. (for example X. diversicaudatum), Longidorus spp. (for example L. elongatus); Hoplolaimus spp. (for example H. coronatus); Aphelenchoides spp. (for example A. ritzemabosi, A. besseyi); and bulb nematodes, such as Ditylenchus spp. (for example D. dipsaci).

The compounds of the invention can be combined with one or more other active pesticidal constituents (for example pyrethroids, carbamates and organophosphates) and/or with attractants, repellents, bactericides, fungicides, nematocides, anthelminthics and so on.

Preferred mixture components are

1. from the group of the phosphorus compounds

acephate, azamethiphos, azinphosethyl, azinphosmethyl, bromophos, bromophosethyl, cadusafos (F-67825), chlorethoxyphos, chlorfenvinphos, chlormephos, chlorpyrifos, chlorpyrifosmethyl, demeton, demeton-S-mathyl, demeton-S-methylsulphone, dialifos, diazinon, dichlorvos, dicrotophos, dimethoate, disulfoton, EPN, ethion, ethoprophos, etrimfos, famphur, fenamiphos, fenitriothion, fensulfothion, fenthion, fonofos, formothion, fostthiazate (ASC-66284), heptenophos, isozophos, isothioate, isoxathion, malathion, methacrifos, methamidophos, methidation, salithion, mevinphos, monocrotophos, naled, omethoate, oxydemeton-methyl, parathion, parathionmethyl, phenthoate, phorate, phosalone, phosfolan, phosphocarb (BAS-301), phosmet, phosphamidon, phoxim, pirimiphos, primiphosethyl, pirimiphosmethyl, profenofos, propaphos, proetamphos, prothiofos, pyraclofos, pyridapenthion, quinalphos, sulprofos, temephos, terbufos, tebupirimfos, tetrachlorvinphos, thiometon, triazophos, trichlorphon, vamidothion;

2. from the group of the carbamates

alancyarb (OK-135), aldicarb, 2-sec-butylphenyl methylcarbamate (BPMC), carbaryl, carbofuran, carbosulfan, cloethocarb, benfuracarb, ethiofencarb, furathiocarb, HC-801, isoprocarb, methomyl, 5-methyl-m-cumenylbutyryl(methyl)carbamate, oxamyl, pirimicarb, propoxur, thiodicarb, thiofanox, 1-methylthioethylideneamino)-N-methyl-N-(morpholinothio)carbamate (UC 51717), triazamate;

3. from the group of the carboxylates

acrinathin, allethrin, alphametrin, 5-benzyl-3-furylmethyl-(E)-(1)-cis-2,2-dimethyl-3-(2-oxothiolan-3-ylidenemethyl)cyclopropanecarboxylate, beta-cyfluthrin, beta-cypermethrin, bioallethrin, bioallethrin((S)-cyclopentylisomer), bioresmethrin, biphenate, (RS)-1-cyano-1-(6-phenoxy-2-pyridyl)methyl-(1RS)-trans-3-(4-tert-butylphenyl)-2,2-dimethylcyclopropanecarboxylate (NCI 85193), cycloprothrin, cyfluthrin, cyhalothrin, cythithrin, cypermethrin, cyphenothrin, deltamethrin, empenthrin, esfenvalerate, fenfluthrin, fenpropathrin, fenvalerate, flucythrinate, flumethrin, fluvaline (D-isomer), imiprothrin (S41311), lambda-cyhalothrin, permethrin, pheothrin ((R)-isomer), prallethrin, pyrethrine (natural products), resmethrin, tefluthrin, tetramethrin, theta-ypermethrin (TD-2344), tralomethrin, transfluthrin, zeta-cypermethrin (F-56701);

4. from the group of the amidines

amitraz, chlordimeform;

5. from the group of the tin compounds

cyhexatin, fenbutatin oxide;

6. others

abamectin, ABG-9008, acetamipirid, Anagrapha falcitera, AKD-1022, AKD-3059, ANS-118, Bacillus thuringiensis, Beauveria bassianea, bensultap, bifenazate (D-2341), binapacryl, BJL-932, bromopropylate, BTG-504, BTG-505, buprofezin, camphechlor, cartap, chlorobenzilate, chlorfenapyr, chlorfluazuron, 2-(4-chlorophenyl)4,5-diphenylthiphene (UBI-T 930), chlorfentezine, chromafenozide (ANS-118), CG-216, CG-217, CG-234, A-184699, 2-naphthylmethyl cyclopropanecarboxylate (Ro12-0470), cyromazin, diacloden (thiamethoxam), diafenthiuron, N-(3,5-dichloro-4-(1,1,2,3,3,3-hexafluoro-1-propoxy)phenyl)carbamoyl)-2-chlorobenzocarboximide, DDT, dicofol, diflubenzuron, N-(2,3-dihydro-3-methyl-1,3-thiazol-2-ylidene)-2,4-xylidine, dinobuton, dinocap, diofenolan, DPX-062, emamectine-benzoate (MK-244), endosulfan, ethiprole (sulfethiprole), ethofenprox, etoxazole (YI-5301), fenazaquin, fenoxycarb, fipronil, flumite (flufenzine, SZI-121), 2-fluoro-5-(4-(4-ethoxyphenyl)-4-methyl-1-pentyl)diphenyl ether (MIT 800), granulosis and nuclear polyhedrosis viruses, fenpyroximate, fenthiocarb, flubenzimine, flucycloxuron, flufenoxuron, flufenprox (IC1-A5683), fluproxyfen, gamma-HCH, halofenozide (RH-0345), halofenprox (MIT-732), hexaflumuron (DE_{—}743), hexythiazox, HOI-9004, hydramethylnon (AC 217300), lufenuron, imidacloprid, indoxacarb (DPX-MP062), kanemite (AKD-2023), M-020, MIT-446, ivermectin, M-020, methoxyfenozide (intrepid, RH-2485), milbemectin, NC-196, neemgard, nitenpyram (TI-304), 2-nitromethyl-4,5-dihydro-6H-thiazine (DS 52618), 2-nitromethyl-3,4-dihydrothiazole (SD 35651), 2-nitromethylene-1,2-thiazinan-3-ylcarbaldehyde (WL 108477), pyripropoxyfen (S-71639), NC-196, NC-1111, NNI-9768, novaluron (MCW-275), OK-9701, OK-9601, OK-9602, propargite, pymethrozine, pyridaben, pyrimidifen (SU-8801), RH-0345, RH-2485, RYI-210, S-1283, S-1833, SB7242, SI-8601, silafluofen, silomadine (CG-177), spinosad, SU-9118, tebufenozide, tebufenpyrad (MK-239), teflubenzuron, tetradifon, tetrasul, thiacloprid, thiocyclam, T1435, tolfenpyrad (OMI-88), triazamate (RH-7988), trifumuron, verbutin, vertalec (mykotal), YI-5301.

Furthermore, it has been observed that the activity of the compounds of the invention can be enhanced by the addition of a synergic or potential-ization agent, for example a synergic agent of the class of oxidase inhibitors, such as piperonylbutoxide or propyl 2-propynylphenyl phosphonate, by the addition of a second compound of the invention or of a pesticidal pyrethroid. When an oxidase-inhibiting synergic agent is present in a composition of the invention, the ratio of the synergic agent to the compound of formula (I) is in the range of 25:1 to 1:25, for example approximately 10:1.

Stabilizers for preventing any chemical degradation which the compounds of the invention may experience are in particular, for example, anti-oxidants (such as tocopherols, butylhydroxyanisole, butylhydroxytoluene), vitamin C (ascorbic acid) and oxygen captors (such as epichlorhydrin) as well as organic and inorganic bases, for example tri-alkylamines such as triethylamine, which can act as basic stabilisers and captors.

The compounds of the present invention have increased pesticidal properties and photostability and/or a reduced toxicity for mammals.

The disclosures in French patent application 97 08 334 from which this application claims priority and in the abstract accompanying this applications are incorporated herein by reference.

### EXAMPLE 1

### 4-[3-3,4-dichlorophenyl)-2-fluoropropyl]-N-2-methylphenyl)-benzamide

Stage A:1-(3,4-dichlorophenyl)-3-(4-bromophenyl)-2-propanone

120 mg of sodium hydride were added at about 15 C-17 C into a solution of 0.5 g of tosmic (tosylmethyl isocyanate), 7.5 cm³ of DMSO and 15 cm³ of ethyl ether. The reaction mixture was agitated for 1 h 45 and 0.62 g of 4-bromobenzyl bromide and 120 mg of sodium hydride were added. A further 7.5 cm³ of DMSO were added and the reaction mixture was poured onto an iced solution of 2N hydrochloric acid, extraction was carried out with ethyl acetate, and washing with sodium carbonate, with water, drying, filtration and concentration. The obtained product was dissolved in a mixture of 5 cm³ of methylene chloride and 10 cm³ of ethyl ether. 2 cm³ of hydrochloric acid were added, followed by concentration and agitation for 5 minutes. Dilution was carried out with an aqueous solution of hydrochloric acid, followed by decantation, washing with an aqueous soda solution, drying, filtration and concentration. The obtained product was chromatographed using silica eluting with the heptaneethyl acetate mixture (8-2). In this way 230 mg of sought product were obtained.

Stage B: 4-[3-(3,4-dichlorophenyl)-2-oxopropyl]-methyl benzoate

A flask containing 3 g of the product prepared in stage A, 600 mg of PdCl₂ (P₃)₂, 18 cm³ of methanol, 3 cm³ of tri-ethylamine and 60 cm³ of DMF (N,N-dimethylformamide) was connected to a carbonylation apparatus. A stream of CO of 900 mbar at 20° C. was passed through, followed by heating to 110° C. and agitation. The reaction mixture was kept under these conditions for a night. The reaction mixture was poured onto a mixture of water, ice and hydrochloric acid. Extraction was carried out 2 times with ethyl acetate. Washing was carried out with water, followed by drying, filtration and concentration. 244 g of product were obtained which were chromatographed on silica eluting with the heptane-ethyl acetate mixture (7-3). In this way 680 mg of sought product were obtained.

STAGE C: 4-[3-(3,4-dichlorophenyl)-2-hydroxypropyl]-methyl benzoate

A mixture of 300 mg of product of stage A, 4 ml of THF (tetrahydrofurane), and 34 mg of sodium borohydride was agitated at 20° C. for 3 hours. The reaction mixture was poured onto a solution of 2N hydrochloric acid, and then extracted with methylene chloride (twice), dried, filtered and evaporated to dryness. In this way 203 mg of sought product were obtained.

STAGE D: 4-[3-(3,4-dichlorophenyl)-2-fluoropropyl)-methyl benzoate

A mixture of 262 mg of product of stage C, 10 ml of methylene chloride, cooled to −70 C and 0.10 ml of diethyl-amino sulphide trifluoride was agitated for 15 minutes. The reaction mixture was poured onto a saturated solution of acid sodium carbonate, followed by extraction with methylene chloride, drying, filtration and evaporation to dryness. 98.3 mg of sought product were obtained.

STAGE E: 4-[3-(3,4-dichlorophenyl)-2-fluoropropyl]-N-(2-methylphenyl)-benzamide

A mixture containing 1 ml of toluene, 0.23 ml of a 2M solution of trimethyl-aluminium in hexane and 61 pi of orthotoluidine was heated to 40 C for 15 minutes. 98 mg of product prepared in stage D and 1 ml of toluene were added drop by drop. The reaction mixture was maintained under agitation for 2 hours. It was poured onto a 2N solution of hydrochloric acid, followed by extraction 2 times with methylene chloride, drying, filtration and evaporation. 102.4 mg of sought product were obtained. M.p.=101 C.

### EXAMPLE 2

### 4-[3-(3,4-dichlorophenyl)-2-fluoro-2-propenyl]-N-(2-methyl phenyl)-benzamide

STAGE A: 3-(3,4-dichlorophenyl)-2-fluoro methyl propenoate

20 g of lithium bromide were introduced at 0 C into 100 cm³ of THF. The obtained solution was cooled to −65 C and 26 cm³ of triethylamine were added. A solution of 10 g of 3,5-dichlorobenzaldehyde and 14.4 g of

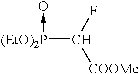

and 50 cm³ of THF was added at 65 C. Agitation was carried out for 1 hour and 40 cm³ of THF were added. Agitation was carried out for 2 hours at 20° C. and the reaction mixture was poured into 400 cm³ of a mixture of water and ice containing 200 mmoles of hydrochloric acid. Extraction was carried out 2 times with ethyl acetate, followed by washing with water, drying, filtration and concentration. 19.8 g of a product were obtained which was chromatographed on silica eluting with the heptane-ethyl acetate mixture (95-5). In this way 1.59 of sought product were obtained.

STAGE B: 3-(3,4-dichlorophenyl)-2-fluoro-2-propen-1-ol

2.5 g of the product of stage A were poured at −70° C. into 40 cm³ of toluene. 15 cm³ of DIBAH were added in 20 minutes. Agitation was carried out at 70° C., for 45 minutes. The temperature was allowed to rise to −20 C. The reaction mixture was poured onto a water-ice-sodium acid phosphate mixture. Ethyl acetate is added followed by agitation. Filtration was carried out, then decanting, drying, filtration and concentration. 1.8 g of sought product were obtained.

STAGE C: 1-3(3-bromo-2-fluoro-1-propenyl)-3,4-dichloro-benzene

A solution containing 0.4 cm³ of phosphorus tribromide in solution in 2 cm³ of carbon tetrachloride was poured in 10 minutes into a solution containing-4.8 g of the product of stage A and 20 cm³ of carbon tetrachloride. The temperature was allowed to rise to 20 C. A further 0.1 cm³ of PBr₃ were added. The reaction mixture was agitated for 1 h 30 and poured into 150 cm³ of iced water. Extraction was carried out with methylene chloride. Filtration and decanting were carried out, washing with a dilute solution of sodium acid carbonate. Decanting, drying, filtration and concentration were carried out. 1.54 g of product were obtained which were chromatographed on silica eluting with the heptane-ethyl acetate mixture (95-5). In this way 1.39 g of sought product were obtained.

STAGE D: 4-[3-(3,4-dichlorophenyl)-3-fluoro-2-propenyl]-benzoic acid

0.3 g of product of stage C was poured into 30 cm³ of dioxan at 20 C. 450 mg of boronic acid were added, then 90 mg of Pd(dba)dba=?₂, then 1.3 g of potassium carbonate. Agitation was carried out for an hour at 20 C. 70 mg of Pd(dba)₂ were added. Heating was carried out for 30 minutes to 100° C. The reaction mixture was returned to 20° C. It was then poured onto a water-ice-hydrochloric acid mixture. Extraction was carried out with ethyl acetate, followed by washing with water, drying, filtration and concentration. The obtained product was taken up into ethyl ether and filtered. Extraction was carried out with an aqueous solution of sodium carbonate, and with water. The aqueous phases were combined and acid-ification was carried out with hydrochloric acid while cooling. Extraction was carried out with ethyl acetate, followed by drying, filtration and concentration. A product was obtained which was taken up with 20 cm³ of a hexane-ethyl acetate mixture (7-3) in reflux. The temperature was returned to 30 C and draining was carried out. The obtained product was chromatographed on silica eluting with the heptane-ethyl acetate-acetic acid mixture (70-30-1). 0.1 g of sought product was obtained.

STAGE E: 4-[3-(3,4-dichlorophenyl)-2-fluoro-2-propenyl]-N-2(-methylphenyl)-benzamide 1 cm³ of oxalyl chloride and 1 drop of DMF were added to a suspension containing 4 cm of methylene chloride at 20° C. A further 0.1 cm³ of oxalyl chloride was added after 1 hour of agitation. The reaction mixture was agitated for another hour and concentrated under reduced pressure (20 mbar) at 40 C. 95 mg of product were obtained which was dissolved in 4 cm³ of methylene chloride at 20° C. 0.1 cm³ of toluidine and 0.1 cm³ of TEA were added. At the end of 1 hour's agitation, dilution is carried out with 75 cm³ of methylene chloride and washing with a 0.5 N solution of iced HCl. Drying, filtration and concentration under reduced pressure were carried out at 40 C. 0.1 g of product was obtained which was made pasty in 10 cm³ of isopropyl ether. The reaction mixture was passed to ultrasounds, carried to reflux, agitated for 1 hour at 0° C. and drained.

92 mg of product were obtained. M.p.=143 C.

### EXAMPLE 3

### 4-[3-(3,4-dichlorophenyl)-3-oxo-1-propenyl]-N-2-methylphenyl)-benzamide

STAGE A: 4-formyl-N-(2-methylphenyl)-benzamide

2 drops of DMF were added at 0° C. into a suspension containing 50.5 g of 4-formylbenzoic acid, and 500 cm³ of methylene chloride. 58 cm³ of oxalyl chloride were added. Agitation was carried out for 30 minutes at 0 C then for 4 hours at 20° C. The reaction mixture was concentrated at 45 C under reduced pressure. It was taken up with 500 cm³ of methylene chloride and the obtained solution was cooled to 0° C. 33 cm³ of pyridine and 43.5 cm³ of orthotoluidine were added. Agitation was carried out for one night at 20 C and hydrolysis with an aqueous solution of 0.5 N hydrochloric acid. Extraction was carried out twice with methylene chloride, followed by combining and drying of the organic phases. Filtration and concentration under reduced pressure were carried out. Chromtography was carried out on silica eluting with the hexane-ethyl acetate mixture (7-3). In this way 63.86 g of sought product were obtained.

STAGE B: 4-[3-(3,4-dichlorophenyl)-3-oxo-1-propenyl]-N-(2-methylphenyl)-benzamide

58 g of product of stage A were dissolved in 1 l of methanol at 20° C. 45.8 g of 3,4-dichloroacetophene were added and in 30 minutes 93 cm³ of a sodium-methanol methylate solution. Agitation was carried out for one night at 20° C. followed by draining. 76.27 g of sought product were obtained.

### EXAMPLE 4

### 4-[3-3,4-dichlorophenyl)-3-hydroxybutyl]-N-2-methylphenyl)-benzamide

Stage A: 4-[3-(3,4-dichlorophenyl)-3-oxopropyl]-N-(2-methylphenyl)-benzamide

0.6 g of palladium on carbon were added at 20° C. into a solution of 1 g of product of the previous example, 30 cm³ of ethyl acetate and 10 cm³ of DMF. This mixture was subjected to a hydrogenation for 45 minutes, then filtered, rinsed with ethyl acetate, and dried. Filtration and concentration followed. Chromatography was carried out on silica eluting with the 90-10 methylene chloride-ethanol mixture. A product was obtained which was dissolved in 25 cm³ of ethyl acetate with reflux. Agitation was carried out for 48 hours at 20° C., followed by draining, concentration and 475 mg of the sought product were obtained, melting at 16 C.

Stage B: 4-3-(3,4-dichlorophenyl)-3-hydroxybutyl]-N-(2-methylphenyl)-benzamide

1 g of product of the previous stage was introduced into 15 ml of THF. The mixture was heated to 40° C. and 0.9 ml of 2M methylmagnesium bromide and 5 ml of THF are added. Heating to 50° C. was carried out for 15 minutes. 0.9 ml of 2M methylmagnesium bromide were added in ethyl ether and 5 ml of THF. Some ml of THF were added and the reaction mixture was maintained at 40° C. for 30 minutes. The temperature was allowed to return to 20° C. The reaction mixture was poured onto iced water and washed with an aqueous solution of ammonium chloride and extracted 3 times with ethyl acetate. The organic phases were combined, dried, filtered and concentrated under reduced pressure at 50 C. 1.02 g of product were obtained which was chromatographed on silica eluting with the heptane-ethyl acetate mixture (7-3). The product was obtained which was concentrated under reduced pressure. 0.56 g of product melting at 121° C. were obtained.

### EXAMPLE 5

### 4-[3-(3,4-dichlorophenyl)-2-butenyl]-N-(2-methylphenyl)-benzamide

A mixture of 0.25 g of product of the previous example, 5 ml of toluene and 0.19 of PTSA (p-toluene sulfonic acid) was heated to 80° C. for 2 hours. The temperature was allowed to return to 20° C. The reaction mixture was poured onto water, and extraction carried out with ethyl acetate; this was followed by drying, filtration and concentration under reduced pressure at 50° C. 0.23 g of product were obtained which was chromatographed on silica eluting with the heptane-ethyl acetate mixture (8-2). In this way 0.18 g of sought product were obtained. M.p.=117 C.

### EXAMPLE 6

### 4-[3-chloro-3-(3,4-dichlorophonyl)-2-propenyl]-N-2-methylphenyl)-benzamide

0.5 g of product of example 4 stage A was introduced into 10 ml of toluene. 1.5 g of phosphorus pentachloride were added. The reaction mixture was heated to 80 C for 2 hours. Agitation was carried out for 2 h 30. The reaction mixture is allowed to return to ambient temperature. The reaction mixture was poured onto acid carbonate of aqueous sodium and maintained under agitation at 20° C. for 1 hour. Extraction was carried out with methylene chloride, followed by drying, filtration and concentration under reduced pressure at 50 C. 0.58 g of product was obtained which is chromatographed (eluant 9-1 heptane-ethyl acetate). After recrystallization in isopropyl ether, 60 mg of product were obtained. M.p.=130° C.

### EXAMPLE 7

### 4-[3-(3,4-dichlorophenyl)-2-propenyl]-N-(2-methylphenyl)-benzamide

STAGE A: 4-[3-(3,4-dichlorophenyl)-3-oxo-1-propenyl]-methyl benzoate

16 g of 3,4-dichloroacetophenone and 13.8 g of 4-formyl methyl benzoate were introduced into 250 ml of methanol. 1.21 g of soda were added and agitation was carried out for one night. The reaction mixture was maintained at 0° C.±5° C., dried, washed and a product was obtained which was recrystallized in ethyl acetate, drained, washed and dried. In this way 22 g of desired product were obtained.

STAGE B: 4-[3-(3,4-dichlorophenyl)-3-hydroxypropyl]-N-(2-methylphenyl)-methyl benzoate

0.8 g of palladium on 10% carbon was poured into a solution containing 2.1 g of product of stage A, 40 cm³ of ethyl acetate and 12 cm³ of DMF. The reaction mixture was placed under hydrogen atmosphere and agitated at 20° C. for 1 h 30. Filtration and washing with ethyl acetate and water were carried out. Drying, filtration and concentration were carried out. 2.47 g of product were obtained which were chromatographed on silica eluting with the heptane-ethyl acetate mixture (8-2). In this way 0.91 g of the sought product were obtained.

STAGE C: 4-[3-(3,4-dichlorophenyl)-2-propenyl]-methyl benzoate

A mixture containing 0.63 g of product of stage B, 20 ml of toluene and 0.2 g of PTSA was heated to 80° C. for 2 hours. The temperature was allowed to return to 20° C. The reaction mixture was poured onto 50 ml of iced water, washed with an aqueous solution of sodium acid carbonate, extracted with ethyl acetate. Drying and filtration were carried out. 0.58 of a sought product is obtained. rf=0.67 (6-4 heptane-ethyl acetate).

STAGE D: 4-[3-(3,4-dichlorophenyl-2-propenyl]-N-(2-methyl-phenyl)-benzamide

1.8 ml of a 2M solution of trimethylaluminium in toluene were added to a solution containing 0.21 ml of ortho-toluidine in 5 ml of toluene and 0.58 g of the product of stage C in 5 ml of toluene. The reaction mixture was heated to 80 C, for 3 hours and allowed to return to 20° C. It was poured onto iced water, acidified by 2N hydrochloric acid up to pH 1. Agitation was carried out for 30 minutes between 0 and 5° C. Extraction was carried out three times with ethyl acetate. Drying, filtration and concentration under reduced pressure were carried out. 0.49 g of sought product was obtained.

### EXAMPLE 8

### 4-[3-(3,4-dichlorophenyl)-2,3-dibromopropyl]-N-2-methylphenyl)-benzamide

0.2 g of product of the previous example was introduced into 10 ml of carbon tetrachloride. The reaction mixture is placed under UV light and 26 μl of bromine and 5 ml of carbon tetrachloride were added. At the end of 2 hours of reaction, the medium was concentrated under reduced pressure. 0.46 g of product was obtained which is chromatographed on silica eluting with the hexane-ethyl acetate mixture (8-2). 0.28 g of product was obtained which was chromatographed on silica eluting with the hexane-ethyl acetate mixture (9-1). In this way 0.26 g of sought product was obtained.

M.p.=175 C.

Using the processes described above, the following products were prepared:

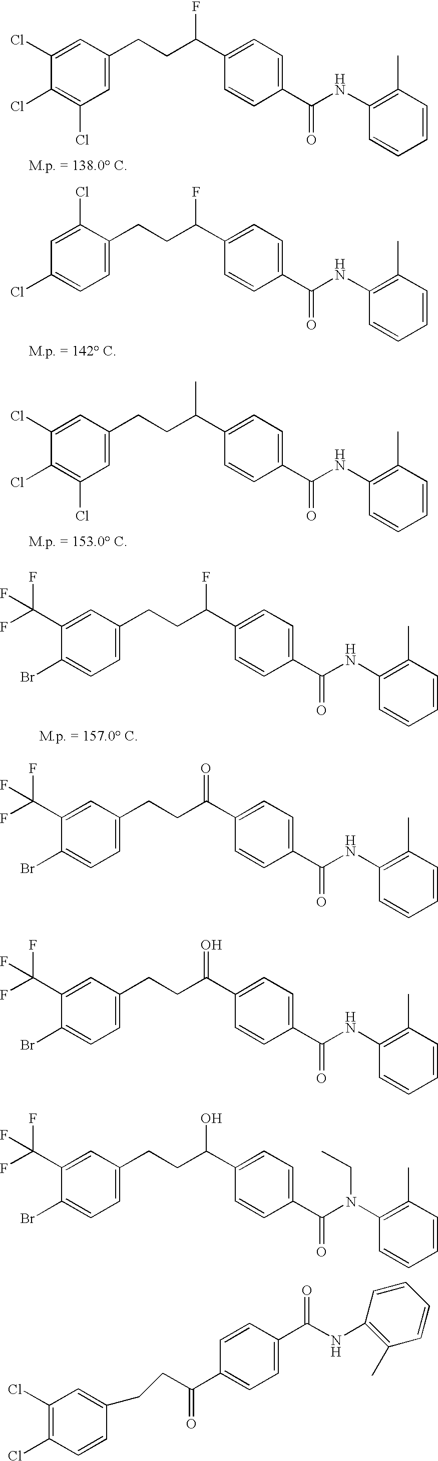

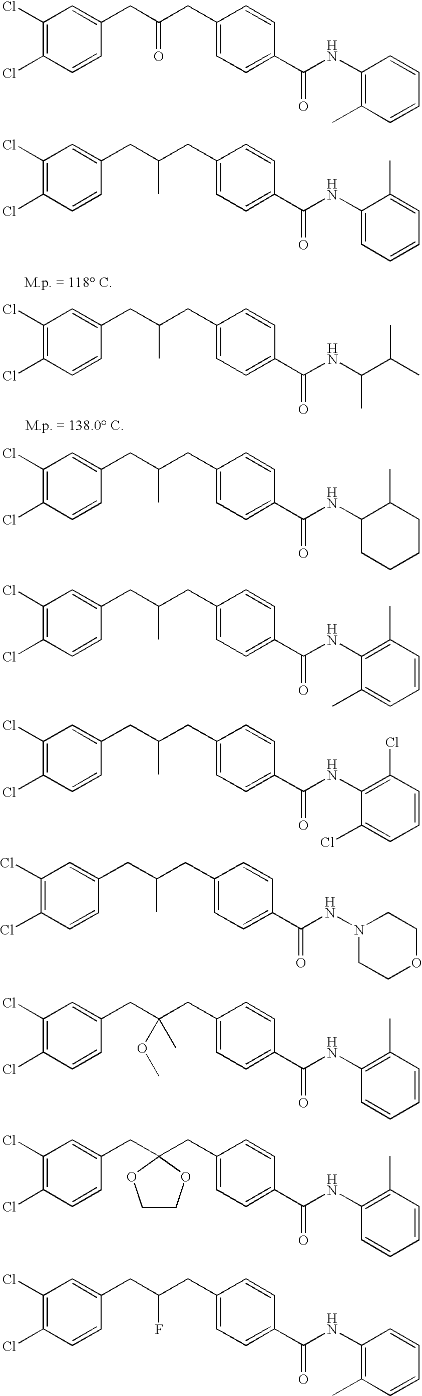

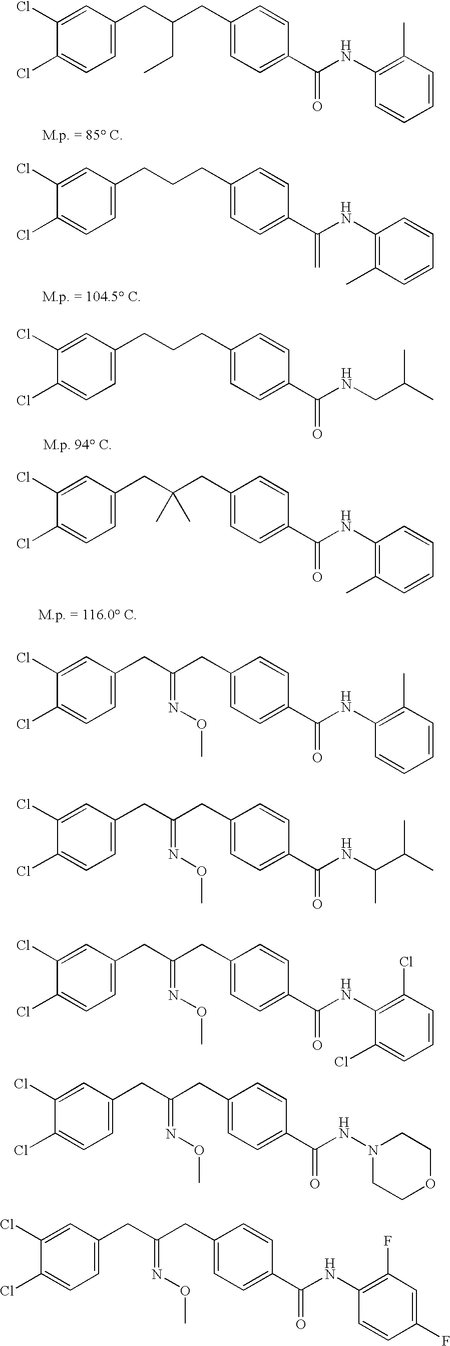

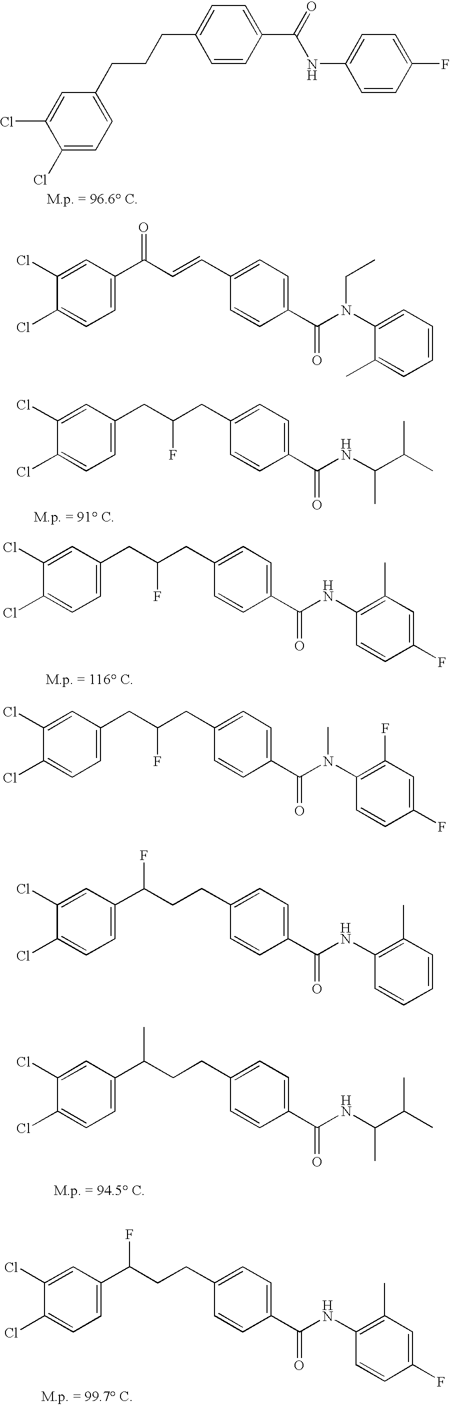

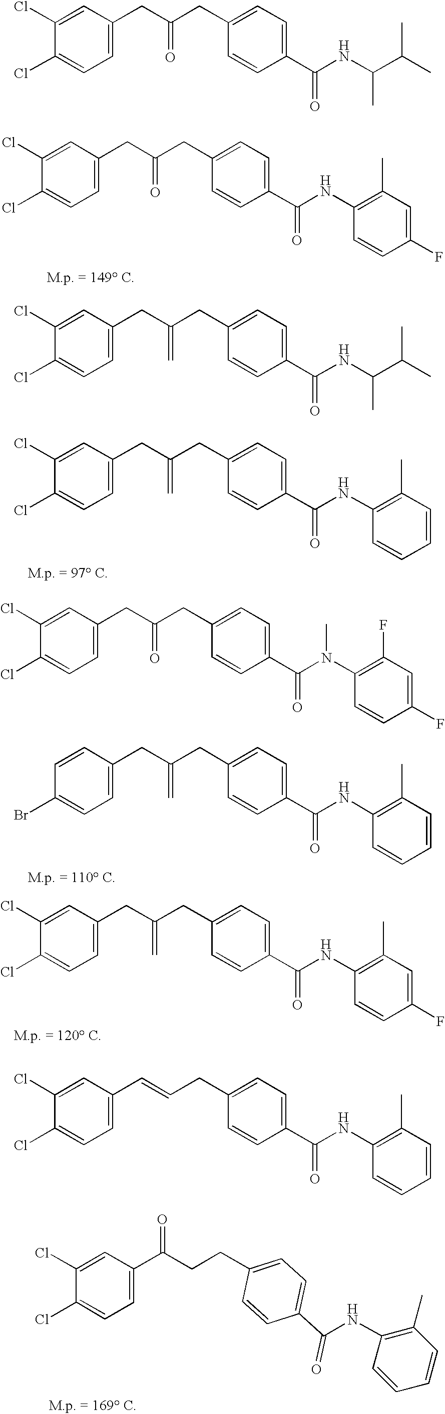

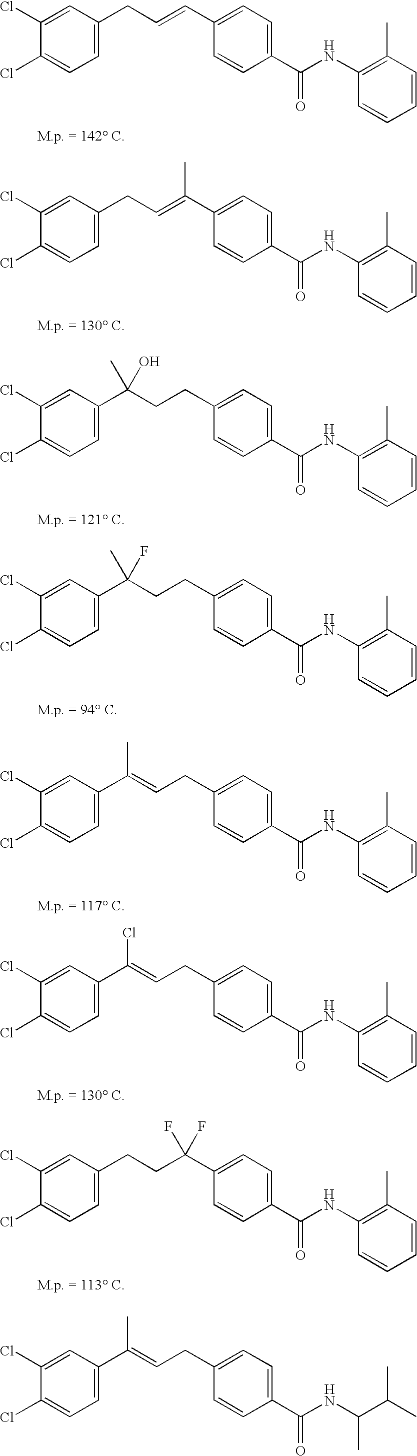

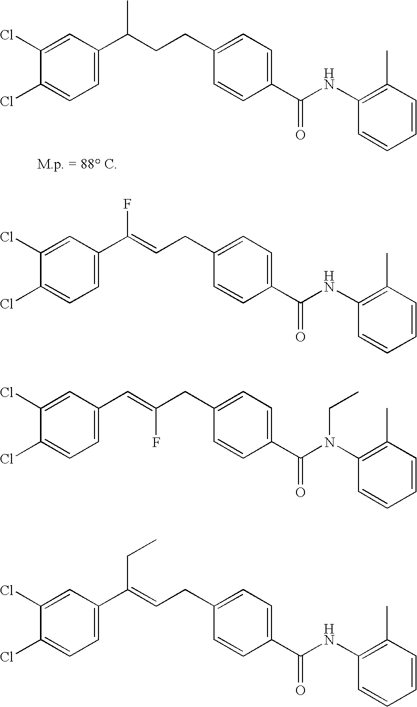

Preparation of Compositions

In the examples of compositions below, the following signs signify:

* Surfactant

# Reacts by forming the polyurea walls of the microcapsules.

| | | |
|---|---|---|
| 1. | Emulsifiable concentrate. | |
| | Active ingredient | 10.00 |
| | Ethoxylated alkylphenol* | 7.50 |
| | Alkylarylsulfonate* | 2.50 |
| | C8-C13 aromatic solvent | 80.00 |
| | | 100.00 |
| 2. | Emulsifiable concentrate. | |
| | Active ingredient | 10.00 |
| | Ethoxylated alkylphenol* | 2.50 |
| | Alkylarylsulfonate* | 2.50 |
| | Ketonic solvent | 64.00 |
| | C8-13 aromatic solvent | 18.00 |
| | Antioxidant | 3.00 |
| | | 100.00 |
| 3. | Wettable powder. | |
| | Active ingredient | 5.00 |
| | C8-13 aromatic solvent | 7.00 |
| | C18 aromatic solvent | 28.00 |
| | Kaolin | 10.00 |
| | Alkylarylsulfonate* | 1.00 |
| | Naphthalenesulfonic acid* | 3.00 |
| | Diatomaceous earth | 46.00 |
| | | 100.00 |
| 4. | Dusting powder. | |
| | Active ingredient | 0.50 |
| | Talc | 99.50 |
| | | 100.00 |
| 5. | Bait. | |
| | Active ingredient | 0.5 |
| | Sugar | 79.5 |
| | Paraffin wax | 20.0 |
| | | 100.00 |
| 6. | Concentrate in emulsion. | |
| | Active ingredient | 5.00 |
| | C8-13 aromatic solvent | 32.00 |
| | Cetyl alcohol | 3.00 |
| | Polyoxyethyleneglycerol monooleate* | 0.75 |
| | Polyoxyethylenesorbitan esters* | 0.25 |
| | Silicon solution | 0.10 |
| | Water | 58.90 |
| | | 100.00 |
| 7. | Concentrate in suspension. | |
| | Active ingredient | 10.00 |
| | Ethoxylated alkylphenol* | 3.00 |
| | Silicon solution | 0.10 |
| | Alkanediol | 5.00 |
| | Fumed silica | 0.50 |
| | Xanthane gum | 0.20 |
| | Water | 80.00 |
| | Buffer | 1.20 |
| | | 100.00 |
| 8. | Microemulsion. | |
| | Active ingredient | 10.00 |
| | Polyoxyethyleneglycerol monooleate* | 10.00 |
| | Alkanediol | 4.00 |
| | Water | 76.00 |
| | | 100.00 |
| 9. | Granules dispersible in water. | |
| | Active ingredient | 70.00 |
| | Polyvinylpyrrolidine | 2.50 |
| | Ethoxylated alkylphenol | 1.25 |
| | Alkylarylsulfonate | 1.25 |
| | Kaolin | 25.00 |
| | | 100.00 |
| 10. | Granules. | |
| | Active ingredient | 2.00 |
| | Ethoxylated alkylphenol* | 5.00 |
| | Alkylarylsulfonate* | 3.00 |
| | C8-13 aromatic solvent | 20.00 |
| | Kieselguhr granules | 70.00 |
| | | 100.00 |
| 11. | Aerosol (aerosol can). | |
| | Active ingredient | 0.30 |
| | Piperonylbutoxide | 1.50 |
| | C8-13 saturated hydrocarbonated solvent | 58.20 |
| | Butane | 40.00 |
| | | 100.00 |
| 12. | Aerosol (aerosol can). | |
| | Active ingredient | 0.3 |
| | C8-13 saturated hydrocarbonated solvent | 10.0 |
| | Sorbitan monooleate* | 1.0 |
| | Water | 40.0 |
| | Butane | 48.7 |
| | | 100.00 |
| 13. | Aerosol (aerosol can). | |
| | Active ingredient | 1.00 |
| | CO2 | 3.00 |
| | Polyoxyethyleneglycerol monooleate* | 1.40 |
| | Propanone | 38.00 |
| | Water | 56.60 |
| | | 100.00 |
| 14. | Lacquer. | |
| | Active ingredient | 2.50 |
| | Resin | 5.00 |
| | Antioxidant | 0.50 |
| | Very aromatic white spirit | 92.00 |
| | | 100.00 |
| 15. | Spray (ready to use). | |
| | Active ingredient | 0.10 |
| | Antioxidant | 0.10 |
| | Odorless kerosene | 99.80 |
| | | 100.00 |
| 16. | Potentiated spray (ready to use). | |
| | Active ingredient | 0.10 |
| | Piperonylbutoxide | 0.50 |
| | Antioxidant | 0.10 |
| | Odorless kerosene | 99.30 |
| | | 100.00 |
| 17. | Microcapsules. | |
| | Active ingredient | 10.0 |
| | C8-13 aromatic solvent | 10.0 |
| | Aromatic diisocyanate # | 4.5 |
| | Ethoxylated alkylphenol* | 6.0 |
| | Alkyldiamine # | 1.0 |
| | Diethylenetriamine | 1.0 |
| | Concentrated hydrochloric acid | 2.2 |
| | Xanthane gum | 0.2 |
| | Fumed silica | 0.5 |
| | Water | 64.6 |
| | | 100.00 |
| 18. | Dispersable concentrate. | |
| | Active ingredient | 5.00 |
| | N-methylpyrrolidinone | 15.00 |
| | N-alkylpyrrolidinone | 53.00 |
| | C8-13 aromatic solvent | 16.00 |
| | Nonylphenol polyoxyethylenic ether phosphate | 6.00 |
| | Ethoxylated alkylphenol | 3.50 |
| | Alkylarylsulfonate | 1.30 |
| | Polyalkyleneglycolic ether | 0.20 |
| | | 100.00 |
| 19. | Soluble concentrate. | |
| | A homogenous mixture is prepared of: | |
| | Active ingredient | 0.25 |
| | Piperonyl butoxide | 1.00 |
| | Tween 80 | 0.25 |
| | Topanol A | 0.10 |
| | Water | 98.40 |
| | | 100.00 |
| 20. | Emulsifiable concentrate. | |
| | An intimate mixture is prepared of: | |
| | Active ingredient | 0.015 |
| | Piperonyl butoxide | 0.50 |
| | Topanol A | 0.10 |
| | Tween 80 | 3.5 |
| | Xylene | 95.885 |
| | | 100.00 |

Biological Study

A) Study on Phaedon cochleariae

The product was dissolved at the desired concentration in an acetone-water mixture (50-50). Foliar disks of Chinese cabbage (Brassica pekinensis) were immersed for five seconds in the solution, then left to dry for one hour. Ten adults (a mixture of males and females) were added into a Petri dish containing a foliar disk each. These were kept at a temperature of 25 C, with a photoperiod of twelve hours. After seven days, the mortality of the insects was checked and the foliar surface consumed was evaluated.

The product of example 6 had a good activity starting from a dose of 300 ppm.

B) Study on Spodoptera littoralis

The product was dissolved at the desired concentration in an acetone-water mixture (50-50). Haricot leaves (Phaseolus vulgaris, var. Delinel) were immersed for five seconds in the solution, then left to dry in a Petri dish for one hour. Ten larvae of Spodoptera littoralis were then added to each dish. These were kept at a temperature of 25 C, with a photoperiod of twelve hours. After seven days, the mortality of the larvae was checked and the foliar surface consumed was evaluated.

The product of examples 1, 7, 2, 5 and 6 had a good activity starting from a dose of 300 ppm.

C) Study on Heliothis virescens

The product was dissolved at the desired concentration in an acetone-water mixture (50-50). 50 μl of solution were deposited on the surface of a small well containing approx-imately 2 grams of plant-based artificial medium. One neonate larva of Heliothis virescens was then introduced into each well, which was sealed with a sheet of cellophane. The tests were kept at a temperature of 25 C, with a photoperiod of twelve hours. The mortality of the larvae was checked after seven days.

The product of examples 1, 7, 2, 5 and 6 had a good activity starting from a dose of 300 ppm.

D) Study on Leptinotarsa decemlineata

The product was dissolved at the desired concentration in an acetone-water mixture (50-50). Foliar disks of aubergine were immersed in the solution, then left to dry for one hour. Ten larvae (3rd stage) were added into a Petri dish each containing a

1. Compounds of formula (1): 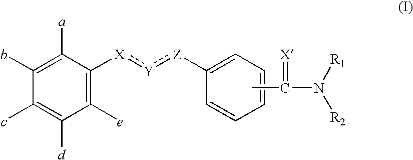
in which
a, b, c, d and e identical to or different from one another, represent a hydrogen atom, a halogen atom, an alkyl, alkenyl or alkynyl, O-alkyl, O-alkenyl or O-alkynyl, S-alkyl, S-alkenyl or S-alkynyl, each radical containing up to 8 carbon atoms, optionally substituted by one or more halogen atoms, a SFs, C N, NO₂ or NH₂ radical, the substituents a, b, c and d being able to form between themselves rings which either carry or do not carry one or more heteroatoms and being able to be substituted;
X and Y or Y and Z identical or different, represent a radical:
—HC═CH—, —(CH₃)C═CH—, -(Hal)C═C—, ═C(f)(g), or ═C═Cx¹x²,
Hal represents a halogen atom;
X¹ representing a hydrogen atom or a halogen atom;
f, g, identical to or different from each other, represent a hydrogen atom, a halogen atom, a free, etherified or esterified hydroxyl radical, an alkyl radical, containing up to 8 carbon atoms, optionally substituted by one or more halogen atoms, or represent a C═O radical, an oxygen atom, foliar disk. These were kept at a temperature of 25 C. After four days, the mortality of the insects was checked.
The product of example 6 had a very interesting activity on this batch starting from a dose of 300 ppm.
X′ represents an oxygen or sulphur atom,
R¹ and R² identical to or different from each other, represent a hydrogen atom, a linear, branched or cyclic, saturated or unsaturated alkyl, CO-alkyl or CO₂-alkyl radical, containing up to 8 carbon atoms, optionally interrupted by one or more heteroatoms, optionally substituted, or an optionally substituted aryl or heteroaryl radical, the —C(X′)-NR¹R² chain being in meta or para position, and
dotted lines representing one or more optional double bonds;
in all their possible isomeric forms as well as their mixture.

2. The compounds of formula (I) defined in claim 1, in the —C(X′)—NR¹R².

3. The compounds of formula (I) defined in claim 1 or 2, in which X′ represents an oxygen atom.

4. The compounds of formula (I) defined in claim 1, 2 or 3, in which R¹ represents a hydrogen atom.

5. The compounds of formula (I) defined in any one of claims 1 to 4, in which R₂ represents an aryl radical, optionally substituted.

6. The compounds of formula (I) defined in any one of claims 1 to 4, in which R₂ represents an alkyl radical containing up to 8 carbon atoms, linear or branched.

7. The compounds of formula (I) defined in any one of claims 1 to 6, in which at least one of the substituents a, b, c, d and e represent a halogen atom.

8. The compounds of formula (I) defined in claim 7, in which the halogen is chlorine or bromine.

9. The compounds of formula (I) defined in claim 7 or 8, in which b, c and d each represent a chlorine or hydrogen atom.

10. The compounds of formula (I) defined in any one of claims 1 to 9, in which a and e each represent a hydrogen atom.

11. The compounds of formula (I) defined in any one of claims 1 to 10, in which Z represents a —CH₂— radical.

12. The compounds of formula (I) defined in any one of claims 1 to 11, in which X represents a —CH₂— radical.

13. The compounds of formula (I) defined in any one of claims 1 to 11, in which X and Y together represent a —HC═CH— radical.

14. The compounds of formula (I) defined in any one of claims 1 to 10 in which Y and Z together represent a —HC═CH— radical.

15. The compounds of formula (I) defined in any one of claims 1 to 11, in which X and Y represent a radical —FC═CH—, —(CH₃C═CH— or —CH═CF—.

16. The compounds of formula (I) the names of which follow:
4-[3-(3,4-dichlorophenyl)-2-fluoropropyl]-N-(2-methyl-phenyl)-benzamide
4-[3-(3,4-dichlorophenyl)-2-fluoropropyl]-N-(1,2-dimethyl-propyl)-benzamide
4-[3 (3,4-dichlorophenyl)-2-fluoropropyl]-N-(4-fluoro-2-methylphenyl)-benzamide
4-[3-(3,4-dichlorophenyl)-3-fluoropropyl]-N-(2-methyl-phenyl)-benzamide
4-[3-(3,4-dichlorophenyl)-2-propenyl]-N-(2-methylphenyl)-benzamide
4-[3-(4-bromo-2-methylphenyl)-2-propenyl]-N-(2-methyl-phenyl)-benzamide
4-[3-(3,4-dichlorophenyl)-2-fluorobutyl]-N-(2-methyl-phenyl)-benzamide
4-[3-(3,4-dichlorophenyl)-2-butenyl]-N-(2-methylphenyl)-benzamide
4-[3-chloro-3-(3,4-dichlorophenyl]-2-propenyl]-N-(2-methylphenyl)-benzamide
4-[3-chloro-3-(3,4-dichlorophenyl]-2-propenyl]-N-(2-methylphenyl)-benzamide
4-[3-(3,4-dichlorophenyl)-2-butenyl]-N-(1,2-dimethyl-propyl]-benzamide
4-[3-(3,4-dichlorophenyl)-2-fluoro-2-propenyl]-N-(2-methylphenyl)-benzamide
4-[3-(3,4-dichlorophenyl)-2-fluoro-2-propenyl]-N-ethyl-N-(2-methylphenyl)-benzamide
4-[3-(3,4-dichlorophenyl)-2-pentenyl]-N-2-methylphenyl)-benzamide.

17. A process for preparing compounds of formula (I) defined in any one of claims 1 to 16, wherein a compound of formula (II): 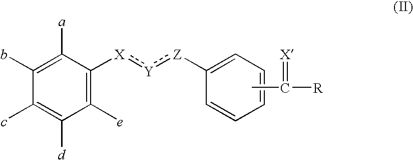
in which a, b, c, d, e, X, Y, Z and X′ and the dotted lines retain the same meaning as in formula (II) in claim 1, and R represents a hydroxy radical, a halogen atom, an alkoxy radical contain-ing up to 4 carbon atoms or a —P(O)(φ)NHφ group in which represents a phenyl group, is subjected to the action of a compound of formula (III):
HNR¹R² (III)
in which R₁ and R₂ have the same meaning as in formula (II) in claim 1 in order to obtain the corresponding compound of formula (I).

18. As chemical products, the compounds of formula (II) defined in claim 17.

19. A process for combating arthropodos and/or helminths and/or molluscs, which comprises the administration to the arthropods and/or helminths and/or molluscs, or to their environment, of a quantity of a compound of formula (I), as defined in any of claims 1 to 16, which is sufficient to destroy the harmful organism.

20. A process for combating and/or eradicating infestations by arthropods and/or helminths and/or molluscs in animals (including humans) and/or plants (including trees) and/or stored products, which comprises the administration to the animal or to the locality of an effective quantity of a compound of formula (I), as defined in any of claims 1 to 16.

21. Compounds of formula (I) to be used in human and veterinary medicine, in public health and/or in agriculture for combating harmful arthropods and/or helminths.

22. A composition comprising:
a) a compound of formula (I), as defined in any of claims 1 to 16,
b) inert excipients suitable for use as pesticides of the said product of formula (I).

23. A composition comprising:
a) a compound of formula (I), as defined in any of claims 1 to 16,
b) inert excipients suitable for use in the veterinary field of the said product of formula (I).
